(19) United States
(12) Patent Application Publication
(10) Pub. No.: US 2016/0185760 A1
(43) Pub. Date: Jun. 30, 2016

(54) PROCESS FOR THE PREPARATION OF MALIC ACID SALT OF SUNITINIB

(71) Applicant: RANBAXY LABORATORIES LIMITED, Delhi (IN)

(72) Inventors: Sudhir Singh SANWAL, Kangra (IN); Saridi Madhava Dileep KUMAR, Gurgaon (IN); Swargam SATHYANARAYANA, Karim Nagar (IN); Rajesh Kumar THAPER, Jammu (IN); Mohan PRASAD, Gurgaon (IN)

(73) Assignee: RANBAXY LABORATORIES LIMITED, New Delhi, Delhi (IN)

(21) Appl. No.: 13/634,737

(22) PCT Filed: Feb. 25, 2011
(86) PCT No.: PCT/IB11/50820

§ 371 Date: Dec. 5, 2012

(30) Foreign Application Priority Data
Mar. 18, 2010 (IN) 635/DEL/2010

(51) Int. Cl. C07D 403/06; C07C 51/41

(52) U.S. Cl. CPC C07D 403/06; C07C 51/41; C07B 2200/07

(57) ABSTRACT

The present invention relates to a process of preparation of malic acid salt of sunitinib.(I).

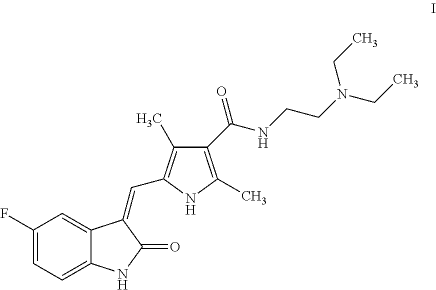 [SMILES: CCN(CC)CCNC(=O)c1c(C)[nH]c(/C=C2\C(=O)Nc3ccc(F)cc32)c1C]

### FIELD OF THE INVENTION

The present invention relates to a process of preparation of malic acid salt of sunitinib.

### BACKGROUND OF THE INVENTION

Sunitinib is chemically described as N-[2-(diethylamino)ethyl]-5-[(Z)-(5 -fluoro-1,2-dihydro-2-oxo-3H-indol-3-ylidine)methyl]-2,4-dimethyl-1H-pyrrole-3-carboxamide as represented by Formula I.

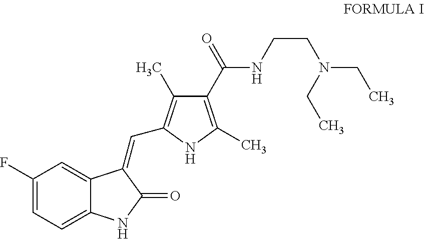 [SMILES: CCN(CC)CCNC(=O)c1c(C)[nH]c(/C=C2\C(=O)Nc3ccc(F)cc32)c1C]

Sunitinib is an oral multi-kinase inhibitor and useful for the treatment of gastrointestinal stromal tumor and advanced renal cell carcinoma. Sunitinib is commercially available as L-malate salt, which is described chemically as butanedioic acid, hydroxy-, (2S)-, compound with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1).

U.S. Pat. No. 7,125,905 describes a process for the preparation of sunitinib base wherein the process involves heating a mixture of N-[2-(diethylamino)ethyl]-5-formyl-2,4-dimethyl-1H-pyrrole-3-carboxamide of Formula II and 5-fluoro-1,3-dihydro-2H-indol-2-one of Formula III in the presence of ethanol and pyrrolidine at 78° C. for 3 hours. The mixture is cooled to room temperature and sunitinib is collected as a base by vacuum filtration.

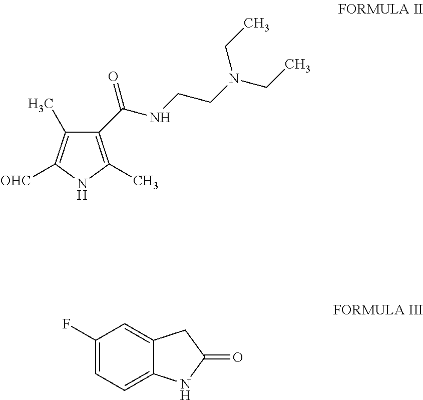 [SMILES: CCN(CC)CCNC(=O)c1c(C)[nH]c(C=O)c1C ; O=C1Cc2cc(F)ccc2N1]

U.S. Publication Nos. 2003/0069298 and 2007/0191458 describe the preparation of crystal Forms I and II of L-malic acid salt of sunitinib from sunitinib base. PCT Publication No. WO 2009/067686 describes processes for preparing crystalline forms of racemic sunitinib malate, sunitinib hemi-L-malate and compositions containing sunitinib base and L- or racemic malic acid from sunitinib base.

WO 2009/150523 describes processes for the preparation of L-malic acid salt of sunitinib, wherein the process involves preparation of L-malic acid salt of N-[2-(diethylamino)ethyl]-5-formyl-2,4-dimethyl-1H-pyrrole-3-carboxamide of Formula II and reacting the salt with 5-fluoro-1,3-dihydro-2H-indol-2-one of Formula III to obtain L-malic acid salt of sunitinib with 75.1% yield.

### SUMMARY OF THE INVENTION

The present inventors have developed a simple and efficient process for the preparation of malic acid salt of sunitinib. The present process does not require the isolation of sunitinib base from the reaction mixture and it can be directly converted into malic acid salt of sunitinib. The present process also avoids the preparation and isolation of malic acid salt of N-[2-(diethylamino)ethyl]-5-formyl-2,4-dimethyl-1H-pyrrole-3-carboxamide of Formula II. The malic acid salt of sunitinib can be obtained by the present process with a yield of about 80% or above directly from the reaction mixture. Thus, the present process minimizes the steps involved in the preparation of sunitinib while it is efficient to obtain malic acid salt of sunitinib with higher yield.

The term “malic acid salt of sunitinib” includes a combination of sunitinib and L-malic acid in any ratio between about 1:0.5 and about 1:1.5.

### DETAILED DESCRIPTION OF THE INVENTION

In one aspect of the present invention is provided a process for the preparation of malic acid salt of sunitinib, wherein the process comprises:
a) reacting N-[2-(diethylamino)ethyl]-5-formyl-2,4-dimethyl-1H-pyrrole-3-carboxamide of Formula II with 5-fluoro-1,3-dihydro-2H-indol-2-one of Formula III in the presence of a solvent to obtain sunitinib base; and
b) treating the reaction mixture obtained in step a) with malic acid to obtain malic acid salt of sunitinib.

N-[2-(diethylamino)ethyl]-5-formyl-2,4-dimethyl-1H-pyrrole-3-carboxamide of Formula II may be prepared according to the method described in, for example, U.S. Pat. No. 7,125,905. N-[2-(diethylamino)ethyl]-5-formyl-2,4-dimethyl-1H-pyrrole-3-carboxamide of Formula II is reacted with 5-fluoro-1,3-dihydro-2H-indol-2-one of Formula III in a solvent to obtain sunitinib base. The reaction may be carried out, for example, by mixing N-[2-(diethylamino)ethyl]-5-formyl-2,4-dimethyl-1H-pyrrole-3-carboxamide of Formula II with the solvent, followed by the addition of 5-fluoro-1,3-dihydro-2H-indol-2-one of Formula III. The solvent may be water, an organic solvent or a mixture thereof. The organic solvent may be an alkanol, for example, n-propanol, methanol, ethanol, isopropanol or n-butanol, an ester, for example, n-butyl acetate, isopropyl acetate, methyl acetate or ethyl acetate, a nitrile, for example, acetonitrile, an aromatic hydrocarbon, for example, toluene, a cyclic ether, for example, tetrahydrofuran, or a ketone, for example, acetone, or a mixture thereof. The reaction mixture may also contain a base. The base may be an organic amine, for example, pyrrolidine. The reaction may be carried out at a temperature of about the boiling point of the solvent. For example, the reaction may be carried out at about 75° C. to about 80° C. when ethanol is used as a solvent. The reaction may be carried out for about 10 minutes to about 10 hours, for example, about 1 hour to about 5 hours. Sunitinib base so obtained need not be isolated from the reaction mixture in any form, solid or oil. The reaction mixture comprising sunitinib base is treated with malic acid to form the malic acid salt of sunitinib. The malic acid may be L-malic acid, D-malic acid, or a mixture thereof. The formation of malic acid salt of sunitinib may be carried out in the same reaction mixture—for example, at substantially the same reaction conditions in which sunitinib base is formed. The malic acid salt of sunitinib may be isolated by filtration, solvent removal, evaporation, solvent precipitation, layer separation, decantation, centrifugation, or a combination thereof.

While the present invention has been described in terms of its specific embodiments, certain modifications and equivalents will be apparent to those skilled in the art and are intended to be included within the scope of the present invention.

### EXAMPLE

### Preparation of L-Malic Acid Salt of Sunitinib

A mixture of N-[2-(diethylamino)ethyl]-5-formyl-2,4-dimethyl-1H-pyrrole-3-carboxamide (1.0 g) and ethanol (12 ml) were stirred. 5-Fluoro-1,3-dihydro-2H-indol-2-one (0.57 g) and pyrrolidine (0.013 g) were added and the reaction mixture was stirred at 78° C. (internal temperature) for 1.5 hours. L-Malic acid (0.37 g) was added to the reaction mixture and the reaction mixture was stirred at 78° C. (internal temperature) for 1 hour. The reaction mixture was cooled to 20° C. to 25° C., filtered under vacuum, washed with ethanol (10 ml) and dried under vacuum at 50° C. for 10 hours to 12 hours to obtain the title compound.

Yield: 80%

We claim:

1. A process for the preparation of malic acid salt of sunitinib, wherein the process comprises:
a) reacting N-[2-(diethylamino)ethyl]-5-formyl-2,4-dimethyl-1H-pyrrole-3-carboxamide of Formula II 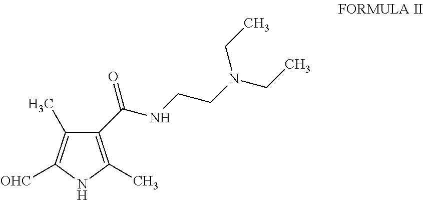 [SMILES: CCN(CC)CCNC(=O)c1c(C)[nH]c(C=O)c1C]
with 5-fluoro-1,3-dihydro-2H-indol-2-one of Formula III 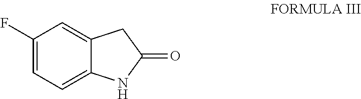 [SMILES: O=C1Cc2cc(F)ccc2N1]
in the presence of a solvent to obtain sunitinib base; and
b) treating the reaction mixture obtained in step a) with malic acid to obtain malic acid salt of sunitinib.

2. A process according to claim 1, wherein the solvent used in step a) is water, an organic solvent, or a mixture thereof.

3. A process according to claim 2, wherein the organic solvent is alkanol, ester, nitrile, aromatic hydrocarbon, cyclic ether, ketone, or a mixture thereof.

4. A process according to claim 3, wherein the organic solvent is alkanol.

5. A process according to claim 4, wherein the alkanol is ethanol.

6. A process according to claim 1, wherein step a) is carried out in the presence of a base.

7. A process according to claim 6, wherein the base is organic amine.

8. A process according to claim 7, wherein the organic amine is pyrrolidine.

9. A process according to claim 1, wherein the sunitinib base formed in step a) need not be isolated from the reaction mixture in any solid or oil form.

10. A process according to claim 1, wherein the malic acid used in step b) is L-malic acid or D-malic acid, or a mixture thereof.
